(19) United States
(12) Patent Application Publication
(10) Pub. No.: US 2008/0167256 A1
(43) Pub. Date: Jul. 10, 2008

(54) Cancer Therapy Via the Inhibition of Skp-2 Expression

(76) Inventors: Hidetoshi Sumimoto, Tokyo (JP); Yutaka Kawakami, Tokyo (JP); Makoto Miyagishi, Tokyo (JP); Kazunari Taira, Ibaraki (JP)

Correspondence Address: MORGAN & FINNEGAN, L.L.P., 3 WORLD FINANCIAL CENTER, NEW YORK, NY, 10281-2101, US

(21) Appl. No.: 11/547,492

(22) PCT Filed: Mar. 22, 2005
(86) PCT No.: PCT/JP2005/005112

§ 371 Date: Sep. 14, 2007

(30) Foreign Application Priority Data
Mar. 31, 2004 (JP) 2004-108560
Sep. 27, 2004 (JP) 2004-280707

(51) Int. Cl. A61K 31/70; C07H 21/04; C12N 15/00

(52) U.S. Cl. 514/44; 536/23.1; 435/320.1

(57) ABSTRACT

The present invention is to provide: a double-stranded RNA (siRNA) capable of suppressing expression of Skp-2 gene, a double-stranded RNA expression cassette capable of expressing a double-stranded RNA, a double-stranded RNA expression vector containing a double-stranded RNA expression cassette, and highly safe therapeutic drug and therapy specific to cancers such as small cell lung cancer having Skp-2 as a molecular target. The RNAi target sequence is set in a plurality of sites in the protein translation region of Skp-2 mRNA, the expressed siRNA, which is a dsRNA exhibiting RNAi effect, is constructed on a lentiviral vector to construct a recombinant viral vector, and various small cell lung cancer cell lines and small cell lung cancers are infected with this viral vector, to confirm the cell proliferation suppression effect in vitro and the proliferation suppression effect in vivo.

### TECHNICAL FIELD

The present invention uses the RNAi (RNA interference) method and relates to: a double-stranded RNA capable of suppressing expression of Skp-2 gene (siRNA: small interfering RNA), a double-stranded RNA expression cassette capable of expressing the double-stranded RNA, a double-stranded RNA expression vector containing the double-stranded RNA expression cassette; a drug for preventing and/or treating a cancer such as small cell lung cancer, which contains these as active components; and a method for preventing and/or treating a cancer such as a small cell lung cancer wherein these are administered, etc.

### BACKGROUND ART

Disorders of cell cycle regulation mechanisms, observed in many malignant tumors, are directly related to uncontrolled proliferation of cancer cells. By suppressing the activity of the cyclin E/cdk2 complex in late G1 phase (late pre-DNA synthetic period) to S phase (DNA synthetic period), p27^{Kip1}, a cyclin-dependent kinase (cdk) inhibitor, inhibits the transition from G1 phase to S phase (for example, see T. Cell, 78: 67-74, 1994). It has been reported that in many malignant tumors including stomach cancer, breast cancer and colon cancer, the decreased expression of p27^{Kip1} is associated with poor prognosis of tumors and very active property of tumors (for example, see Cancer Res., 62: 3819-3825, 2002). As the level of p27^{Kip1} protein is mainly regulated by ubiquitin-proteasomal proteolysis, the enhanced degradation of p27^{Kip1} is considered to be an important factor of the decreased expression of p27^{Kip1} in malignant tumors (for example, see Nature, 396: 177-180). Skp-2 (S-phase kinase-associated protein 2 (p45)), a member of F-box protein family, is a specific substrate-recognition subunit of SCF ubiquitin-protein ligase complex, and is involved in the degradation of p27^{Kip1} (for example, see Nature Cell Biol. 1: 207-214, 1999). The increased expression of Skp-2 has been reported in many cancers including small cell lung cancer (SCLC) (for example, see American J. Pathol. 161: 207-216, 2002), oral squamous cell cancer (for example, see Proc. Natl. Acad. Sci., USA, 98: 5043-5048, 2001), lymphoma (for example, see Proc. Natl. Acad. Sci., USA, 98: 2515-2520, 2001), or stomach cancer (for example, see Cancer Res., 62: 3819-3825, 2002). The p27^{Kip1} levels in these cancers were decreased by contraries. It is suggested that the increased expression of Skp-2 could be involved in the decreased level of p27^{Kip1} protein in these tumors. In 44% of SCLC cases, the increased expression of Skp-2 gene was observed during the genetic amplification of 5p11-1 region (amplicon), and it was accompanied by the decreased expression of p27^{Kip1} (for example, see American J. Pathol. 161: 207-216, 2002).

In the developmental process of living organisms, cells differentiate into cells having various traits, while their proliferation, life and death are stringently controlled. Also in adults which have gone through the developmental stages, proliferation, differentiation, and apoptosis of each cell are stringently controlled to maintain the homeostasis as an individual. In other words, the destiny of each cell is controlled by the accurate transmission of extracellular signals including hormones, neutrotransmitters, cell growth factors and cytokines into the cell through receptors on the cell membrane. The mechanism that transmits the extracellular signals to the nuclei in cells and controls genetic information is called intracellular signaling mechanism, and the continuous reaction of intracellular protein interaction in this mechanism is called intracellular signaling pathway. The intracellular signaling pathway transmits signals by repeating the process of becoming an activated form by receiving signals from upstream, and becoming an inactivated form again after transmitting the signals downstream.

The mitogen-activated protein kinase (MAPK) pathway, one of the intracellular signaling systems, plays an important role in the cell proliferation/differentiation signals. MAPK is a phosphoenzyme of protein whose molecular weight is about 40000, and forms a phosphorylation cascade, which is MAP kinase kinase kinase (MAPKKK)→MAP kinase kinase (MAPKK)→MAP kinase (MAPK), in various cell types of eukaryotes. This cascade becomes activated at downstream of ras, a proto-oncogene, and not only works as a cell proliferation signal, but also induces cell differentiation, cell proliferation arrest or increased cell motility. Further, as MAPK-based constant hyperfunction is observed in many cancer cells, it is considered that the specific inhibition of such hyperfunction will lead to anticancer therapies.

With regard to the MAPK pathway, point mutation of BRAF, one of MPKKKs, is also detected frequently (66%) in malignant melanomas, and the association with cancerous changes is suggested. All mutations are observed in the activated region of kinase domain or in the region adjacent thereto. Based on the analysis of some mutations which have occurred frequently (V599E, L596E, G463V, G468A), it has been reported that the kinase activity of BRAF is increased by mutation, and that ERK is activated as a result, and further, that the mutated BRAF has the transformation ability of NIH3T3 cells (for example, see Nature, 417: 949-954, 2002). Furthermore, the increased MAPK activity has been reported constantly in many malignant melanoma cell lines and malignant melanoma tissues (for example, see Cancer Research, 63, 756-759, 2003). These reports suggest that the mutated BRAF is an oncogene closely related to the development of malignant melanoma, and that it can be a molecular target for the treatment of malignant melanoma. However, in the assay system mentioned above, the function of excessive amount of the mutated BRAF, which is far beyond the physiological expression level, is detected, and therefore, the effect of endogenous mutated BRAF on MAPK and the association with the cancerous changes remains to be elucidated.

In addition, it has been also reported that the increased expression of Skp-2 and the BRAF mutation are commonly observed in many cancers including lung cancer, oral squamous cell cancer, lymphoma, stomach cancer, colon cancer, malignant melanoma, brain tumor, colon cancer, lung cancer, ovarian cancer, sarcoma, thyroid cancer (for example, see Cancer Res., 62: 3819-3825, 2002; American J. Pathol. 161: 207-216, 2002; Proc. Natl. Acad. Sci., USA, 98: 5043-5048, 2001; Proc. Natl. Acad. Sci., USA, 98: 2515-2520, 2001; Nature, 417, 949-954, 2002).

On the other hand, it has been found that in a certain living organism (Caenorhabditis elegans), gene expression can be specifically inhibited by a double-stranded RNA (dsRNA) (for example, see the pamphlet of WO99/32619; Nature, 391, 806-811, 1998). This phenomenon is called RNAi (RNA interference), a phenomenon in which dsRNA consisting of a sense RNA and an antisense RNA homologous to a certain gene disrupts the homologous part of the transcript (mRNA) of the gene. Thereafter, this phenomenon has been found in lower eucaryotic cells including various animals (for example, see Cell, 95, 1017-1026, 1998; Proc. Natl. Acad. Sci. USA, 95, 14687-14692, 1998; Proc. Natl. Acad. Sci. USA, 96, 5049-5054, 1999) and plants (for example, see Proc. Natl. Acad. Sci. USA, 95, 13959-13964, 1998).

Initially, it was thought to be difficult to use RNAi for mammalian cells when RNAi was discovered because of the following reason: when dsRNAs larger than about 30 bp are introduced into mammalian cells, the non-specific inhibition of gene expression induced by interferon responses occurs, and as a result, the specific inhibition of gene expression caused by RNAi is no longer observed. However, it was shown in 2000 that RNAi could occur even in early mouse embryos and mammalian cultured cells, and it was revealed that the inducing mechanism of RNAi itself exists in mammalian cells as well (for example, see the pamphlet of WO01/36646; FEBS Lett, 479, 79-82, 2000).

It is obviously useful if it is also possible to inhibit the expression of a particular gene or gene cluster in mammals by using the function of RNAi thus described. As a number of diseases (cancers, endocrine diseases, immune diseases, etc.) are triggered when a particular gene or gene cluster is expressed abnormally in mammals, the inhibition of gene or gene cluster can be used for treating these diseases. In addition, diseases may develop due to the expression of mutated proteins, and in these cases, it becomes possible to treat such diseases by inhibiting the expression of mutated alleles. Further, it is said that the gene-specific inhibition thus described can be used also for treating, for example, viral diseases caused by retroviruses such as HIV (viral genes in retroviruses are expressed by being integrated into the genomes of their hosts.).

With regard to dsRNA that induces the function of RNAi, it was initially believed to be necessary to introduce dsRNA larger than about 30 bp into cells. However, it has been revealed recently that shorter (21 to 23 bp) dsRNA (siRNA: small interfering RNA) can induce RNAi without showing cytotoxicity even in mammalian cell lines (for example, see Nature, 411, 494-498, 2001). SiRNA is recognized as a powerful tool to suppress gene expression in all developmental stages of somatic cells. Further, in progressive genetic diseases, etc., it is a promising method for suppressing expression of genes that act as a causative gene of such diseases before the development thereof.

### DISCLOSURE OF THE INVENTION

### Object to be Attained by the Invention

The object of the present invention is to provide: a double-stranded RNA (siRNA) capable of suppressing expression of Skp-2 gene by using RNAi, a double-stranded RNA expression cassette capable of expressing a double-stranded RNA, a double-stranded RNA expression vector containing a double-stranded RNA expression cassette, and highly safe therapeutic drug and therapy specific to cancers such as small cell lung cancer having Skp-2 as a molecular target.

### Means to Attain the Object

Skp-2 is involved in the degradation of a plurality of cell cycle regulators such as p27^{Kip1}, p21, or c-myc, etc. It is considered that the increased expression of Skp-2 in many cancers is one of the important mechanisms of cell cycle regulation disorder. Therefore, for the purpose of elucidating the role of Skp-2 in the development of SCLC and examining whether Skp-2 could be a good molecular target for cancer therapies, the present inventors have attempted to analyze changes in the characteristics of cancers by conducting virus-mediated Skp-2-specific RNA interference (RNAi). Lentivirus-mediated Skp-2-specific RNAi suppressed the endogenous Skp-2 protein level of ACC-LC-172, a small cell lung cancer cell line with increased expression of Skp-2, and at the same time, decreased the in vitro proliferation activity. Though the RNAi-mediated suppression of Skp-2 protein level correlated with the increase in p27^{Kip1} and p21, it did not induce the inactivation of myc transcriptional activity. Similarly, adenovirus-mediated Skp-2-specific RNAi suppressed the in vivo proliferation of ACC-LC-172 subcutaneous tumor. These results indicate the possibility that RNAi of Skp-2 would be a useful method for gene therapy of cancers. Further, the present inventors have previously proposed a cancer therapy with the use of a double-stranded RNA (siRNA) capable of suppressing expression of BRAF gene such as mutated BRAF (V599E) gene by using the RNAi method (Japanese Patent Application No. 2004-124485). They have found that when mutated BRAF (V599E)-specific RNAi and Skp-2-specific RNAi are used simultaneously for malignant melanoma cell lines exhibiting BRAF mutation and the increased expression of Skp-2, cell proliferation and cell invasive ability are significantly suppressed in comparison to where each RNAi is used alone. The present invention has been completed based on the above-mentioned findings.

In other words, the present invention relates to: “1” a double-stranded RNA capable of suppressing expression of Skp-2 gene, which consists of a sense strand RNA and an antisense strand RNA homologous to a specific sequence of Skp-2 mRNA, which is a target of the double-stranded RNA; “2” the double-stranded RNA according to “1”, wherein the specific sequence of SKp-2 mRNA, which is a target of the double-stranded RNA consists of a RNA corresponding to the base sequence shown by SEQ ID NO: 2 of the sequence listing and a complementary sequence of the RNA; “3” the double-stranded RNA according to “1”, wherein the specific sequence of Skp-2 mRNA which is a target of the double-stranded RNA consists of a RNA corresponding to the base sequence shown by SEQ ID NO: 3 of the sequence listing and a complementary sequence of the RNA; and “4” the double-stranded RNA according to any one of “1” to “6”, wherein the specific sequence of SKp-2 mRNA which is a target of the double-stranded RNA is a base sequence of 19 to 24 bp.

The present invention also relates to: “5” a double-stranded RNA expression cassette capable of expressing the double-stranded RNA according to any one of “1” to “4”, which consists of a sense strand DNA—a linker—an antisense strand DNA of a specific sequence of Skp-2gene; “6” the double-stranded RNA expression cassette according to “5”, which consists of the base sequence shown by SEQ ID NO: 4 of the sequence listing; “7” the double-stranded RNA expression cassette according to “5”, which consists of the base sequence shown by SEQ ID NO: 5 of the sequence listing; “8” a double-stranded RNA expression vector wherein the double-stranded RNA expression cassette according to any one of “5” to “7” is connected to downstream of a promoter; and “9” the double-stranded RNA expression vector according to “8”, which is an HIV lentiviral vector or an adenoviral vector.

The present invention further relates to: “10” a suppressor of Skp-2 gene expression comprising the double-stranded RNA according to any one of “1” to “4”, the double-stranded RNA expression cassette according to any one of “5” to “7”, or the double-stranded RNA expression vector according to “8” or “9”, as an active component; “11” a drug for preventing and/or treating a cancer which comprises the double-stranded RNA according to any one of “1” to “4”, the double-stranded RNA expression cassette according to any one of “5” to “7”, or the double-stranded RNA expression vector according to “8” or “9”, as an active component; “12” the drug for preventing and/or treating a cancer according to “11”, wherein the cancer results from overexpression of Skp-2 gene or is accompanied by overexpression of Skp-2 gene; “13” the drug for preventing and/or treating a cancer according to “11”, wherein the cancer is a small cell lung cancer (SCLC); “14” a method for suppressing Skp-2 gene expression wherein the double-stranded RNA according to any one of “1” to “4”, the double-stranded RNA expression cassette according to any one of “5” to “7”, or the double-stranded RNA expression vector according to “8” or “9” is introduced into a living body, a tissue or a cell; “15” a method for preventing and/or treating a cancer wherein the double-stranded RNA according to any one of “1” to “4”, the double-stranded RNA expression cassette according to any one of “5” to “7”, or the double-stranded RNA expression vector according to “8” or “9” is introduced into a living body, a tissue or a cell; “16.” the method for preventing and/or treating a cancer according to “15”, wherein the cancer results from mutation or overexpression of Skp-2 gene or is accompanied by overexpression of Skp-2 gene; “17” the method for preventing and/or treating a cancer according to “15”, wherein the cancer is a small cell lung cancer (SCLC); “18” a method for preventing and/or treating a cancer wherein the double-stranded RNA according to any one of “1” to “4”, the double-stranded RNA expression cassette according to any one of “5” to “7”, or the double-stranded RNA expression vector according to “8” or “9” is introduced into a living body, a tissue or a cell; “19” the method for preventing and/or treating a cancer according to “15”, wherein the cancer results from mutation or overexpression of Skp-2 gene or is accompanied by overexpression of Skp-2 gene; “20” the method for preventing and/or treating a cancer according to “18”, wherein the cancer is a small cell lung cancer (SCLC); “21” a method for preventing and/or treating a cancer wherein the following (1) and (2) are introduced into a living body, a tissue or a cell:

(1) the double-stranded RNA according to any one of “1” to “4”, the double-stranded RNA expression cassette according to any one of “5” to “7”, or the double-stranded RNA expression vector according to “8” or “9”;
(2) a double-stranded RNA capable of suppressing the expression of mutated BRAF gene, which consists of a sense strand RNA and an antisense strand RNA homologous to a specific sequence of BRAF mRNA, which is a target of the double-stranded RNA, a double-stranded RNA expression cassette capable of expressing the double-stranded RNA, which consists of a sense strand DNA—a linker—an antisense strand DNA of a specific sequence of BRAF gene, or a double-stranded RNA expression vector wherein the double-stranded RNA expression cassette is connected to downstream of a promoter.

### EFFECT OF THE INVENTION

As it can be seen from the results of Examples, it is revealed that the specific suppression of Skp-2 expression by RNA interference method leads to the accumulation of p27^{Kip1} and the arrest of cell cycle in G1 phase, and brings about a powerful suppressing effect on proliferation. Consequently, drugs for preventing/treating cancers of the present invention, etc., are promising as a useful drug for gene therapy since they have a cell proliferation suppressing effect in vitro and a proliferation suppressing effect in vivo. In addition, a Skp-2-specific siRNA of the present invention exhibited little proliferation suppressing effect on cells with low Skp-2 expression levels (such as 293T cells, fibroblasts). Therefore, a Skp-2-specific siRNA of the present invention is expected to have an effect selectively working on cancer cells, and thereby is expected to be used for highly safe molecular-targeted therapies. Further, these siRNAs are highly useful not only for the application to the medical field but also as a tool for basic research on cell cycle regulation and disorders thereof. Furthermore, the simultaneous use of mutated BRAF (V599E)-specific RNAi and Skp-2-specific RNAi is useful as a highly safe molecular-targeted cancer therapies showing overexpression of both mutated BRAF (V599E) gene and Skp-2 gene.

### BEST MODE OF CARRYING OUT THE INVENTION

The double-stranded RNA of the present invention is not particularly limited as long as it is a double-stranded RNA capable of suppressing expression of Skp-2 gene and consists of a sense strand RNA and an antisense strand RNA homologous to a specific sequence of Skp-2 mRNA, which is a target of the double-stranded RNA. Though the origin of the Skp-2 gene is not particularly limited, a human-derived Skp-2 gene is preferable. As the Skp-2 gene, Skp-2 gene consisting of the base sequence shown by SEQ ID NO: 1 of the sequence listing is exemplified.

The specific sequence of Skp-2 mRNA which is a target of the double-stranded DNA mentioned above means a partial sequence of a specific region in Skp-2 mRNA, preferably a partial sequence whose base length is 19 to 24 bp. As the target sequence of Skp-2 mRNA, a sequence specific to Skp-2 mRNA is preferable. As the target sequence of Skp-2 mRNA, a double-stranded RNA consisting of RNA derived from the base sequence ATCAGATCTCTCTACTTTA shown by SEQ ID NO: 2 of the sequence listing (19 mer from 949^{th} to 967^{th} of the base sequence shown by SEQ ID NO: 1) and its complementary sequence, and a double-stranded RNA consisting of RNA derived from the base sequence AGGTCTCTGGTGTTTGTAA shown by SEQ ID NO: 3 of the sequence listing (19 mer from 408^{th} to 426^{th} of the base sequence shown by SEQ ID NO: 1) and its complementary sequence are specifically exemplified.

In addition, the sense strand RNA homologous to a specific sequence of Skp-2 mRNA which is a target of the double-stranded RNA means, for example, RNA derived from the DNA sequence shown by SEQ ID NO: 2 or 3 mentioned above, and the antisense strand RNA homologous to a specific sequence of Skp-2 mRNA which is a target of the double-stranded RNA means RNA complementary to the sense strand RNA mentioned above. The double-stranded RNA of the present invention is usually constructed as siRNA wherein these sense strand RNA and antisense strand RNA are bound together. However, a double-stranded RNA constructed as siRNA of a mutated sense strand RNA sequence, which is a sequence wherein one or a few bases are deleted, substituted, or added in a sense strand RNA sequence, and of a mutated antisense strand RNA sequence complementary to the mutated sense strand RNA sequence, is also within the scope of the present invention, for convenience. The above-mentioned “base sequence wherein one or a few bases are deleted, substituted, or added” means a base sequence wherein any number of bases, for example, 1 to 5 bases, preferably 1 to 3 bases, more preferably 1 to 2 bases, still more preferably 1 base, is deleted, substituted, or added.

In order to construct the double-stranded RNA (dsRNA) of the present invention, known methods such as a method using synthesis and a method using genetic recombination technique can be used appropriately. With the method using synthesis, a double-stranded RNA can be synthesized by ordinary methods based on sequence information. In addition, with the method using genetic recombination technique, a double-stranded RNA could be constructed by the process comprising the following steps: constructing an expression vector in which a sense strand DNA or an antisense strand DNA is incorporated; introducing the vector into a host cell; then obtaining a sense strand RNA and an antisense strand RNA produced by transcription, respectively. However, it is preferable to construct a desired double-stranded RNA by the process comprising the following steps: constructing a double-stranded RNA expression cassette consisting of a sense strand DNA—a linker—an antisense strand DNA of a specific sequence of Skp-2 gene; and connecting the double-stranded RNA expression cassette to downstream of a promoter of the expression vector for in vivo expression/construction.

As the double-stranded RNA expression cassette of the present invention mentioned above consisting of a sense strand DNA—a linker—an antisense strand DNA of a specific sequence of Skp-2 gene, the followings are specifically exemplified: a double-stranded RNA expression cassette consisting of the base sequence ATCAGATCTCTCTACTTTA TTCAAGAGA TAAAGTAGAGAGATCTGAT ttttt shown by SEQ ID NO: 4 of the sequence listing and having TTCAAGAGA as a linker sequence, and the double-stranded RNA expression cassette consisting of the base sequence CTCTGGTGTTTGTAATTCAAGAGA TTACAAACACCAGAGACCT ttttt shown by SEQ ID NO: 5 of the sequence listing. These double-stranded RNA expression cassettes can form a double-stranded RNA consisting of a sense strand RNA corresponding to a sense strand DNA and an antisense strand RNA corresponding to an antisense DNA, when they are transcribed in host cells.

Further, as the expression vector capable of inserting a double-stranded RNA expression cassette in downstream of a promoter, for example, murine leukemia retroviral vectors (Microbiology and Immunology, 158, 1-23, 1992), adeno-associated viral vectors (Curr. Top. Microbiol. Immunol., 158, 97-129, 1992), adenoviral vectors (Science, 252, 431-434, 1991), liposomes, etc., are specifically exemplified. However, the use of HIV lentiviral vectors having a characteristic that long-term expression can be efficiently realized even in non-dividing cells, and of adenoviral vectors capable of introducing genes in vivo with high viral titer can be considered. Furthermore, these expression systems may contain a regulatory sequence that not only causes expression but also regulates expression. Introduction of a double-stranded RNA expression cassette into these expression vectors can be performed by ordinary methods. For example, the double-stranded RNA expression vector of the present invention can be constructed by inserting a double-stranded DNA consisting of a sense strand DNA—a linker sequence—an antisense strand DNA of a sequence complementary to the target mRNA into downstream of an appropriate promoter (U6 promoter, etc.) of these expression vectors.

With regard to the suppressor of Skp-2 gene expression of the present invention and the drug for preventing and/or treating cancers of the present invention, there is no particular limitation as long as it contains (1) the above-mentioned double-stranded RNA of the present invention, the double-stranded RNA expression cassette of the present invention, or the double-stranded RNA expression vector of the present invention as an active component. Further, with regard to the suppressor of Skp-2 gene and mutated BRAF (V599E) gene expression of the present invention and the drug for preventing and/or treating cancers of the present invention, there is no particular limitation as long as it contains the above-mentioned (1) double-stranded RNA of the present invention, the double-stranded RNA expression cassette of the present invention, or the double-stranded RNA expression vector of the present invention, and (2) a double-stranded RNA capable of suppressing expression of mutated BRAF gene, which consists of a sense strand RNA and an antisense strand RNA homologous to a specific sequence of BRAF mRNA which is a target of the double-stranded RNA, a double-stranded RNA expression cassette capable of expressing the double-stranded RNA, which consists of a sense strand DNA—a linker—an antisense strand DNA of a specific sequence of BRAF gene, or a double-stranded RNA expression vector wherein the double-stranded RNA expression cassette is connected to downstream of a promoter, as active components. For the introduction or administration of these expression suppressors and drugs for preventing and/or treating cancers into living bodies, tissues, cells, etc. of mammals, they can be used with various compounding ingredients for prescription such as pharmaceutically acceptable carriers, binders, stabilizers, excipients, diluents, pH buffers, disintegrators, solubilizers, auxiliary solubilizers, isotonic agents, etc., which are generally used in this field. The pharmaceutical compositions used with the pharmaceutically acceptable carriers can be formulated in a form known by itself in the pharmaceutical field in accordance with the administration form, for example, oral (including intraoral or sublingual) administration, or parenteral administration (injection, etc.).

As to the method for suppressing Skp-2 gene expression of the present invention and the method for preventing and/or treating cancers of the present invention, there is no particular limitation as long as it is a method wherein (1) the above-mentioned double-stranded RNA of the present invention, the double-stranded RNA expression cassette of the present invention, or the double-stranded RNA expression vector of the present invention is introduced into a living body, a tissue or a cell of a mammal. As to the method for suppressing Skp-2 gene and mutated BRAF (V599E) gene expression of the present invention and the method for preventing and/or treating cancers of the present invention, there is no particular limitation as long as it is a method wherein the above-mentioned (1) double-stranded RNA of the present invention, the double-stranded RNA expression cassette of the present invention, or the double-stranded RNA expression vector of the present invention, and (2) a double-stranded RNA capable of suppressing the expression of mutated BRAF gene, which consists of a sense strand RNA and an antisense strand RNA homologous to a specific sequence of BRAF mRNA which is a target of the double-stranded RNA, a double-stranded RNA expression cassette capable of expressing the double-stranded RNA, which consists of a sense strand DNA—a linker—an antisense strand DNA of a specific sequence of BRAF gene, or a double-stranded RNA expression vector wherein the double-stranded RNA expression cassette is connected to downstream of a promoter are introduced into a living body, a tissue or a cell of a mammal. As a method for introducing such double-stranded RNA, double-stranded RNA expression cassette, or double-stranded RNA expression vector into a living body, a tissue or a cell of a mammal, oral or parenteral administration methods are exemplified. For example, it can be administered orally by generally used administration forms for instance, in a dosage form such as powders, granules, capsules, syrups, suspensions, etc., or it can be administered parenterally by injection in a dosage form such as solutions, emulsions, suspensions, etc., and in addition, it can be administered intranasally in a form of spray. Further, the dosage can be selected appropriately according to the kinds of diseases, body weight of patients, administration forms, etc.

As the cancer to be the target of the drug for preventing/treating cancers of the present invention, or the method for preventing/treating cancers of the present invention, a cancer results from overexpression of Skp-2 gene or is accompanied by overexpression of Skp-2 gene is exemplified, and more specific examples include malignant melanoma, colon cancer, lung cancer, breast cancer, ovarian cancer, brain tumor, and thyroid cancer in addition to small cell lung cancer. Further, as the cancer to be the target of the drug for preventing/treating cancers of the present invention, or the method for preventing/treating cancers of the present invention, a cancer wherein the overexpression of Skp-2 gene and the mutation of BRAF gene are commonly observed is exemplified, and more specific examples include lung cancer, oral squamous cell cancer, lymphoma, stomach cancer, colon cancer, malignant melanoma, brain tumor, colon cancer, lung cancer, ovarian cancer, sarcoma, and thyroid cancer.

As the above-mentioned double-stranded RNA capable of suppressing the expression of mutated BRAF (V599E) gene, which consists of a sense strand RNA and an antisense strand RNA homologous to a specific sequence to be targeted by BRAF mRNA, a mutated BRAF gene derived from human is preferable, and as the mutated BRAF gene, genetic DNAs of mutated BRAF, indicated as V599E, L596E, G463V, G468A, are specifically exemplified, and the mutated BRAF (V599E) gene consisting of a base sequence shown by SEQ ID NO: 10 of the sequence listing (a mutated gene wherein 1857^{th} T is substituted with A in a BRAF gene), which is deeply involved in the development of malignant melanoma, is particularly preferably exemplified.

The specific sequence to be targeted by BRAF mRNA mentioned above means a partial sequence of a specific region in BRAF mRNA, preferably a partial sequence whose base length is 19 to 21 bp. As the target sequence of BRAF mRNA, a sequence specific to BRAF mRNA is preferable, and a target sequence containing a mutated site of mutated BRAF mRNA is particularly preferable. As the target sequence containing a mutated site of mutated BRAF mRNA, a double-stranded RNA consisting of RNA containing a mutated site of mutated BRAF (V599E) gene and derived from the base sequence GCT ACA GaG AAA TCT CGA T shown by SEQ ID NO: 11 of the sequence listing (19 mer from 1850^{th} to 1868^{th} of the base sequence shown by SEQ ID NO: 10) and its complementary sequence is specifically exemplified. In addition, as a target sequence capable of suppressing the expression of BRAF mRNA, though it is not a specific sequence containing a mutated site of mutated BRAF gene, a double-stranded RNA consisting of RNA derived from the base sequence GCC ACA ACT GGC TAT TGT TA shown by SEQ ID NO: 12 of the sequence listing (20 mer from 1624^{th} to 1643^{rd} of the base sequence shown by SEQ ID NO: 10) and its complementary sequence, and a double-stranded RNA consisting of RNA derived from the base sequence shown by SEQ ID NO: 13 of the sequence listing (21 mer from 1669^{th} to 1689^{th} of the base sequence shown by SEQ ID NO: 10) and its complementary sequence are specifically exemplified.

As the double-stranded RNA expression cassette mentioned above consisting of a sense strand DNA—a linker—an antisense strand DNA of a specific sequence of BRAF gene, the followings are specifically exemplified: a double-stranded RNA expression cassette consisting of the base sequence GCT ACA GaG AAA TCT CGA T TTCAAGAGA ATC GAG ATT TCt CTG TAG C ttttt shown by SEQ ID NO: 14 of the sequence listing and having TTCAAGAGA as a linker sequence, and the double-stranded RNA expression cassette consisting of the base sequence GCC ACAACT GGC TAT TGT TA TTCAAGAGA TA ACA ATA GCC AGT TGT GGC ttttt shown by SEQ ID NO: 15 of the sequence listing, and the double-stranded RNA expression cassette consisting of the base sequence GTA TCA CCA TCT CCA TAT CAT TTCAAGAGA ATG ATA TGG AGA TGG TGA TAC ttttt shown by SEQ ID NO: 16 of the sequence listing. These double-stranded RNA expression cassettes can form a double-stranded RNA consisting of a sense strand RNA corresponding to a sense strand DNA and an antisense strand RNA corresponding to an antisense DNA, when they are transcribed in host cells.

In addition to the above, the meaning of the sense strand RNA homologous to a specific sequence to be targeted by BRAF mRNA is the same as in the case of Skp-2 mentioned above. The method for constructing a BRAF double-stranded RNA (dsRNA) and the method for constructing an expression vector capable of inserting a double-stranded RNA expression cassette into downstream of a promoter are the same as in the case of Skp-2 mentioned above.

The present invention will be described more specifically with reference to Examples, but the technical scope of the present invention is not limited to these exemplifications. All of the statistical analyses were conducted in accordance with unpaired Student-t test.

### EXAMPLE 1

### Cell Lines

An ACC-LC-172 cell line which had been established from a Japanese patient of small cell lung cancer (provided by Dr. Takahashi, Aichi Cancer Center) was maintained in RPMI 1640 (Sigma) supplemented with 10% (v/v) fetal bovine serum, penicillin and streptomycin. 293T cells and 8 kinds of malignant melanoma cell lines (Skmel23, A375, 888, 397, 526, 624, 928, 1363) were purchased from American Type Culture Collection (ATCC), and maintained in DMEM (Sigma) supplemented with 10% (v/v) fetal bovine serum, penicillin and streptomycin.

### EXAMPLE 2

### HIV Vector

The HIV vector for expressing siRNA was constructed based on HIV-U6i-GFP plasmid as described previously (J. Gene Med., 2004). In brief, HIV-U6i-GFP has two expression units. One is an siRNA expression cassette, an expression unit in which a short hairpin RNA is transcribed from a human U6 promoter, and another is a GFP expression cassette, an expression unit in which a GFP gene is transcribed from a CMV promoter. For the purpose of expressing siRNA, a complementary oligonucleotide annealed in vitro for target sequences was inserted into two BspMI sites downstream of a human U6 promoter.

(siRNA Expression Lentiviral Vector Targeting Skp-2)

In Skp-2 mRNA, two sites of siRNA target sequences were selected; (S2) ATCAGATCTCTCTACTTTA and (S5) AGGTCTCTGGTGTTTGTAA. Two complementary oligonucleotides cacc-(target sense)-TTCAAGAGA-(target antisense)-TTTT and gcatAAAAA-(target sense)-TCTCTTGAA-(target antisense) were synthesized for each target sequence, and annealed in vitro. This double-stranded (ds) oligonucleotide has 5′-protruding ends complementary to two BspMI sites existing in downstream of U6 promoter in an HIV-U6i-GFP plasmid, and thereby it can be easily inserted into an siRNA expression cassette in an HIV-U6i-GFP. GL3B siRNA (siRNA against firefly luciferase) HIV vector, which was a control vector, was also constructed with the target sequence GTGCGCTGCTGGTGCCAAC (SEQ ID NO: 6). A mutation-specific anti-BRAF siRNA HIV vector (target; GCTACAGAGAAATCTCGATGG; SEQ ID NO: 7) for mRNA of BRAF (Skp-2) that frequently mutates in melanoma was used as a control in a reporter assay. From these HIV vectors, siRNA is formed by the following process: a sense strand and an antisense strand form loop structure at the position of the linker sequence (TTCAAGAGA) to form a short hairpin RNA; then the linker is removed by Dicer. In the construction of HIV vectors, 293T cells were transfected with HIV plasmid vectors, pMD.G (VSV-G env expression plasmid), pMDLg/p.RRE (a third-generation packaging plasmid) and pRSV Rev (Rev expression plasmid) (the latter two plasmids were provided by cell Genesys) by calcium phosphate transfection.

The culture supernatants were collected 48 hours later, and used as viral vectors after concentration. The viral titer was calculated based on GFP expression measured in 293T cells after infection.

(siRNA Expression Lentiviral Vector Targeting BRAF)

In BRAF (V599E) RNA, the siRNA target sequence (#1′) GCTACAGAGAAATCTCGAT (SEQ ID NO: 11) targeted at a sequence which contains a V599E mutated site and wherein the 8^{th} base is mutated from T to A, was selected. As with the case of Skp-2, the synthetic nucleotide consisting of cDNA with 19 to 21 base length homologous to the target mRNA sequence (sense strand), a linker sequence, cDNA complementary to the sense strand (antisense strand), and the transcription stop signal TTTTT, was inserted into the BspMI site downstream of a U6 promoter, and a unit capable of expressing the sense strand—linker—antisense strand RNA homologous to the target mRNA sequence was constructed from the U6 promoter. This RNA forms a loop in the linker part after it is transcribed in a cell, and forms a stem structure between the sense strand and the antisense strand, and it becomes siRNA after the linker part is excised by Dicer in the cytoplasm. From these HIV vectors, siRNA is formed by the following process: a sense strand and an antisense strand form a loop structure at the position of the linker sequence (TTCAAGAGA) to form a short hairpin RNA; then the linker is removed by Dicer. In the construction of HIV vectors, 293T cells were transfected with HIV plasmid vectors, pMD.G (VSV-G env expression plasmid), pMDLg/p.RRE (the third-generation packaging plasmid) and pRSV Rev (Rev expression plasmid) by calcium phosphate transfection. The culture supernatants were collected 48 hours later, and used as viral vectors after concentration. The viral titer was calculated based on GFP expression measured in 293T cells after infection.

### EXAMPLE 3

### In Vitro Proliferation Inhibition Assay

ACC-LC-172 cells (1×10⁵ cells) were infected with the siRNA HIV vectors specific to Skp-2 (S2 or S5) or firefly luciferase (GL3B) at 100 MOI (multiplicity of infection). The number of the cells was counted every 3 days by trypan blue dye exclusion method until day 9. 293T cells (3×10⁴ cells) were infected with siRNA HIV vectors specific to control GL3B or Skp-2 (S5) at 100 MOI, and the number of the cells was counted every 3 days until day 9. Three kinds of malignant melanoma cell lines (624mel, A375mel, 526mel), 5×10⁴ cells each, were infected with 4 kinds of HIV lentiviral vectors, control GL3B, BRAF siRNA #1′, Skp-2 siRNA S5 or BRAF/Skp-2 siRNA, at 100 MOI, and the number of the cells was counted every 3 days by trypan blue dye exclusion method until day 6 or 9 (n=3).

### EXAMPLE 4

### Western Blot Analysis

Proteins used for Western blotting were extracted from the cultured cells at day 9 used in the in vitro proliferation inhibition assay with the use of the protein lysate having the following compositions (20 mM Tris-HCl (pH 7.5), 12.5 mM β-glycerophosphoric acid, 2 mM EGTA, 10 mM NaF, 1 mM benzamide, 1% NP-40, protease inhibition cocktail (complete, EDTA-free (Rosh)), 1 mM Na₃VO₄). Prior to the protein extraction, it was confirmed by flow cytometry that the GFP expression was comparable among the treatment groups, and thus it was confirmed that the relativity of gene introduction efficiency was maintained. The concentrations of the proteins were quantitated with DC protein assay kit (Bio-Rad). As primary antibodies, anti-p45 Skp-2 antibody (Zymed Laboratories), anti-actin antibody (Sigma), anti-p27^{Kip1} antibody (BD Transduction), anti-Rb antibody (Cell Signaling), anti-p21 antibody (Santa Cruz), anti-BRAF antibody, anti-ERK2 antibody, anti-ppERK2 antibody were used. As a secondary antibody, HRP-conjugated anti-IgG antibody was used, and enzyme reactions were detected with the use of Super Signal West Femto Maximum Sensitivity Substrate (Pierce).

### EXAMPLE 5

### Cell Cycle Assay

The cells used in the in vitro proliferation inhibition assay were collected at day 9, and stained by using Cycle TEST PLUS DNA Reagent Kit (Becton Dickinson). The stained cells were analyzed with FACS Calibur (Becton Dickinson), and subsequently the state of cell cycle was analyzed with ModFit software (Becton Dickinson).

### EXAMPLE 6

### Construction of hTERT Reporter

A 0.4 kb human telomerase riverse transcriptase (hTERT) promoter sequence was amplified by genomic PCR with the use of the following primer set; the forward primer CGCTGGGGCCCTCGCTGGCGTCCCT (nts −324 to −300, numbered based on the translation initiation site; SEQ ID NO: 8); and the reverse primer: CAGCGGCAGCACCTCGCGGTAGTGG (nts +48 to +72; SEQ ID NO: 9). The reaction conditions were as follows: after denaturation for 4 minutes at 95° C., 27 cycles of denaturation for 1 minute at 95° C., annealing for 1 minute at 70° C. and extension for 1 minute at 72° C. were performed, and then at 72° C. for 7 minutes to complete the reaction. Next, the PCR product was subcloned into a pCRII vector of TA Cloning kit (Invitrogen). After confirming the correct sequence, the translation initiation codon was mutated from ATG to TTG by using QuickChange site-directed mutagenesis kit (STRATAGENE). Finally, the hTERT promoter was subcloned into a pGL3-basic vector (Promega) (pGL3-hTERT). The pGL3-hTERT expresses a firefly luciferase gene under the control of the 0.4 kb hTERT promoter.

### EXAMPLE 7

### Reporter Assay

ACC-LC-172 cells (5×10⁵ cells), which had been infected with HIV vectors and thereby stably expressing siRNA specific to Skp-2 (S5) or to BRAF, were transfected with 1 μg of Renilla luciferase expression plasmid, pRL-SV40 (Promega), and 1 μg of one of the following firefly luciferase expression plasmids, pGL3-hTERT, pGL3-Basic or pGL3-control (Promega), by using Lipofectamine (Invitrogen). 48 hours after the transfection, the cells were collected and the luciferase activity was analyzed with Dual-Glo Luciferase Assay System (Promega) and a Berthold luminometer. Each firefly luciferase activity was normalized to Renilla luciferase activity.

### EXAMPLE 8

### Adenovirus for siRNA

Adenoviral vectors containing Ad5/35 chimeric fiber protein (Gene, 285: 69-77, 2002) were used. The vector plasmid pAdHM34 and the shuttle vector plasmid pHMCMV-GHP1 were described previously in the paper (Cancer Res., 61: 7913-7919, 2001). pHMCMV-GFP1 contains a CMV promoter, a GFP gene derived from pEGFP-N1 (Clontech) and a bovine growth hormone (BGH) poly (A) signal. The siRNA expression unit containing a human U6promoter and 2 BspMI cloning sites were excised from the HIV-U6i-GFP plasmid by EcoRI treatment, and then subcloned into the EcoRI site, which was located downstream of the BGH poly (A) signal in pHMCMV-GHP1. This vector was designated as pHMCMV-GFP-U6i. The ds oligonucleotides specific to the short hairpin RNA can be directly subcloned into the BspMI site of the pHMCMV-GFP-U6i as with the case of the HIV-U6i-GFP. As a result, the shuttle vector plasmids containing ds oligonucleotides specific to Skp-2 (S5) or GLB3 were constructed. The adenoviral vectors, AdF35-Skp-2 siRNA S5 and AdF35-GL3B, were constructed by the in vitro ligation method as described previously (Hum. Gene Ther., 9: 2577-2583, 1998). Both adenoviral vectors were amplified with 293 cells, and the viral titers were measured with Adeno-X Rpaid Titer Kit (Clontech).

### EXAMPLE 9

### Animal Experiment

Male NOD/SCID mice of 6 weeks of age (Japan Clea) were subcutaneously inoculated with 5×10⁶ ACC-LC-172 cells. About 1 week after the implantation, when the largest tumor diameter reached about 3 to 4 mm, 1×10⁸ ifu (infectious unit) of AdF35-Skp-2 siRNA S5 or AdF35-GL3B was injected into the tumor (day 0). The injection of adenovirus was further repeated twice every 2 days. The tumor volume (largest diameter×perpendicular diameter×height) was measured every 2 or 3 days until day 13. The animal experimental protocol was approved by the Committee for Animal Experimentation of Keio University School of Medicine. Mice were treated according to the guidelines by the Committee for Animal Experimentation of Keio University.

### EXAMPLE 10

### HIV Vector-Mediated Skp-2-Specific siRNA Expression Against Small Cell Lung Cancer Cell Lines Highly Expressing Skp-2 Gene Induced In Vitro Cell Proliferation Inhibitory Effect

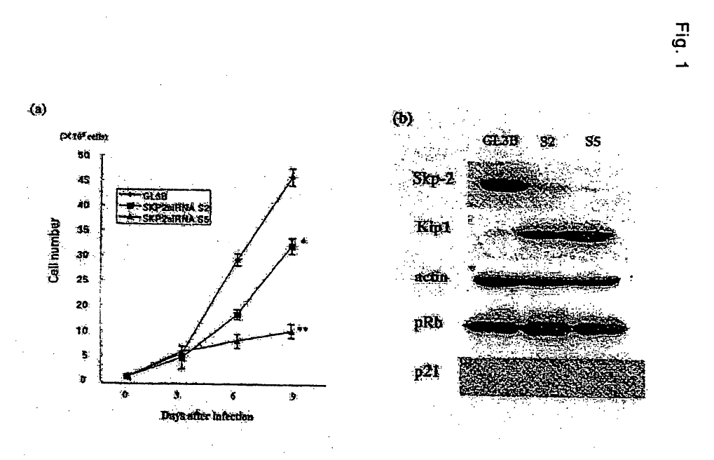

The present inventors have constructed siRNA-expressing HIV vectors targeting Skp-2 mRNA, infected a small cell lung cancer cell line, ACC-LC-172, which shows the increased expression of the Skp-2 gene with the vector, and then evaluated the RNAi effect by analyzing the Skp-2 protein level with Western blotting. From among these siRNA HIV vectors, 2 HIV vectors, S2 and S5, which show an excellent Skp-2 RNAi effect, were used in the following researches. By infecting ACC-LC-172 cells with these HIV vectors and a control siRNA HIV vector specific to firefly luciferase (GL3B), the correlation between the Skp-2 RNAi effect and in vitro cell proliferation was analyzed. The gene introduction efficiency monitored by GFP was comparable among the treatment groups (98.7 to 99.9%). It has been found that the in vitro cell proliferation rate of ACC-LC-172 cells infected with the S5 siRNA HIV vector was significantly lowered in comparison to that of the cells infected with the GL3B siRNA HIV vector (P<0.0001) (FIG. 1a). The in vitro cell proliferation rate of ACC-LC-172 cells infected with the S2 siRNA HIV vector was greater than that of the cells infected with S5, but significantly lower than that of the cells infected with GLB3 (P=0.0005) (FIG. 1a). By Western blot analysis of Skp-2 protein collected 9 days after the infection, it has been revealed that the degree of decrease in Skp-2 protein level is correlated with the in vitro cell proliferation inhibitory effect (FIGS. 1a and 1b). In contrast, the p27^{Kip1} protein level was increased in the cells infected with the S2 and S5 siRNA HIV vectors. Interestingly, the increase in p27^{Kip1} exhibited an inverse correlation with the in vitro cell proliferation rate and the Skp-2 protein level (FIG. 1b). The other cdk inhibitor, p21, increased in a same pattern as that of p27^{Kip1}, in the cells infected with S2 and S5 siRNA HIV vectors (FIG. 1b). In this cell line, the p57^{Kip2} protein was below the detection limit. The Rb protein level was comparable among these cells. In the cell cycle analysis conducted 9 days after the infection, it was observed that the percentage of S and G2/M phase of the cells infected with S5 siRNA HIV vector (44.6%) was lower than that of the cells infected with GLB3 siRNA HIV vector (57.1%). As described above, in ACC-LC-172 cells, Skp-2 RNAi induced the increased levels of both P27^{Kip1} and p21 proteins, the decrease in dividing cell populations, and the inhibition of in vitro cell proliferation.

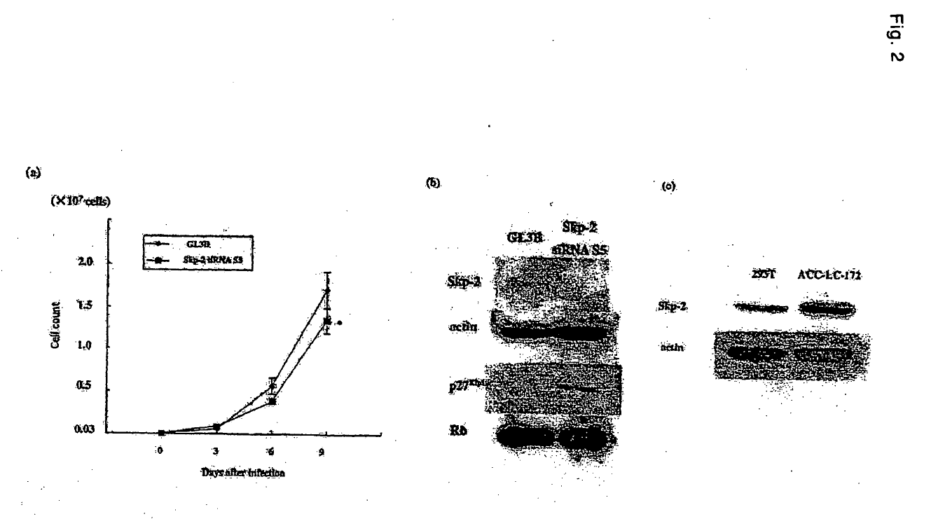

On the other hand, 293T cells without the increased Skp-2 expression exhibited greater resistance to the in vitro cell proliferation suppressing effect of the siRNA HIV vector specific to Skp-2 (S5) (P=0.1835) (FIG. 2a). In the Western blot analysis of Skp-2 and p27^{Kip1} proteins, the comparable results were observed between 293T cells and ACC-LC-172 cells, however, 293T cells exhibited less prominent results (FIG. 2b). The basic level of Skp-2 protein was lower in 293T cells than in ACC-LC-172 cells (FIG. 2c), suggesting the possibility that it would be a cause of the resistance of 293T cells to the proliferation inhibition by Skp-2 RNAi.

### EXAMPLE 11

### Myc-Transcriptional Activity is not Involved in the Cell Proliferation Inhibitory Effect of Skp-2 RNAi on ACC-LC-172 Cells

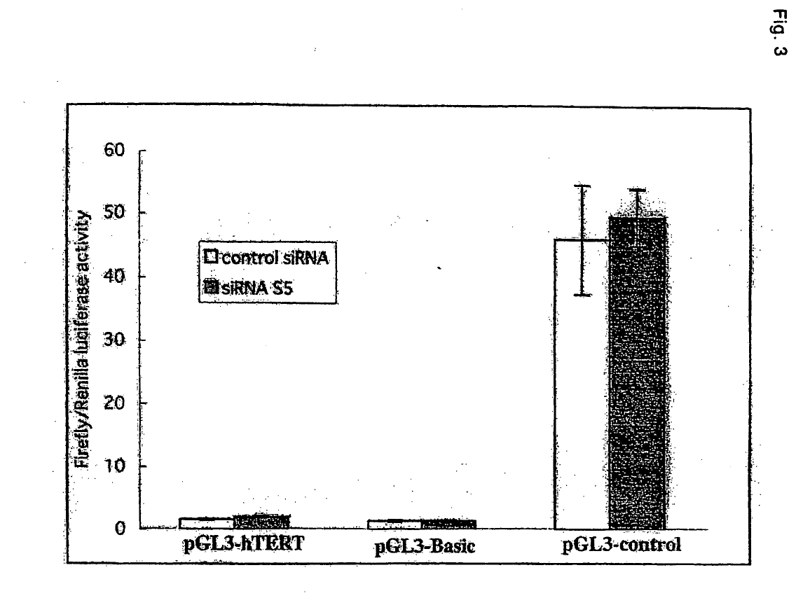

According to recent reports, it is suggested that Skp-2 mediates the ubiquitination of myc protein other than p27^{Kip1}, and at the same time, acts as a transcription cofactor of c-myc (Mol. Cell, 11: 1177-1188, 2003; Mol. Cell, 11: 1189-1200, 2003). As myc mediates the activation of cyclin E-cdk2 and cyclin D-cdk4, and promotes G1/S transition, the present inventors have examined the possibility that the suppression of transcriptional activity of myc would be involved in the cell proliferation inhibitory effect by Skp-2 RNAi. The overexpression of myc mRNA has been reported in many SCLCs (Lung Cancer 34: S43-46, 2001), and mild increase in the number of c-myc copies was also observed in ACC-LC-172 cells (2.03-fold). When the firefly luciferase expression vector (pGL3-hTERT) having an hTERT promoter (containing 2 sites of myc-binding sequence E-box (CACGTG)) upstream thereof was transfected to an ACC-LC-172 cell line, the firefly luciferase activity normalized by Renilla luciferase activity was only slightly increased (1.1- to 2.7-fold) in comparison to the activity of control pGL3-Basic, indicating that myc transcriptional activity was relatively weak in that cell line (FIG. 3). In ACC-LC-172 cells in which Skp-2 was knocked down by the S5 vector, the decrease in firefly luciferase activity caused by pGL3-hTERT was not observed, indicating that myc transcriptional activity was not involved in the proliferation inhibitory effect by Skp-2 RNAi.

### EXAMPLE 12

### Adenoviral Vector Expressing siRNA Specific to Skp-2

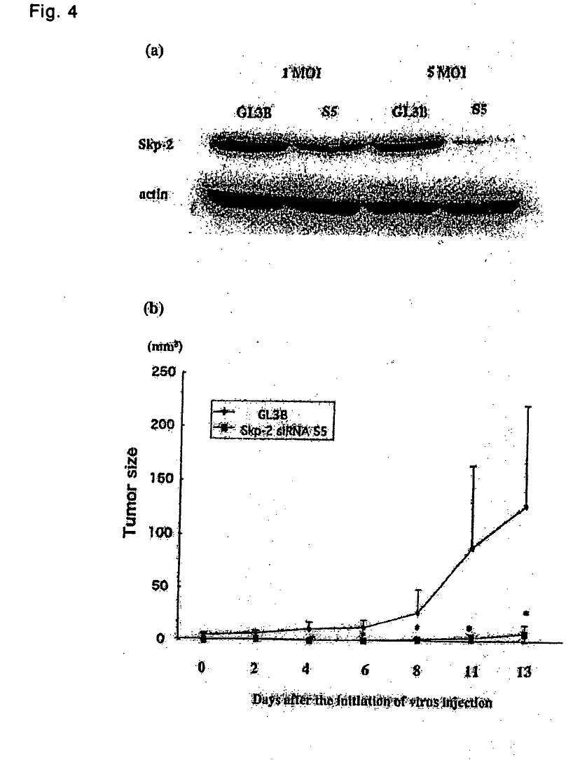

An Skp-2-specific siRNA expression adenoviral vector was constructed because, in comparison to HIV vectors, adenoviral vectors can easily adjust viruses with high titer and are excellent in gene introduction efficiency in vivo. The Skp-2 protein level of ACC-LC-172 cells infected with the adenoviral vector AdF35-Skp-2 siRNA S5 at 5 MOI was significantly decreased in comparison to control cells infected with AdF35-GL3B (FIG. 4), and in vitro cell proliferation inhibition was accompanied.

### EXAMPLE 13

### Adenovirus-Mediated Skp-2 RNAi Decreased the Tumorigenic Potential of ACC-LC-172 Cells In Vivo

Next, whether adenovirus-mediated Skp-2-specific RNAi can suppress the proliferation of ACC-LC-172 cells in vivo was examined. siRNA adenoviral vectors specific to Skp-2 (S5) or control GL3B were inoculated 3 times every 2 days to subcutaneously transplanted tumors established in NOD/Scid mice, and the tumor volume was measured in time course. 13 days after the first intratumoral injection of the virus, tumor proliferation was significantly inhibited in ACC-LC-172 cells infected with S5, in comparison to the case of the cells infected with GL3B (FIG. 4b) (P<0.05). These results suggested that Skp-2 is an excellent target for the treatment of cancers with the increased Skp-2 expression level.

### EXAMPLE 14

### Analysis of Skp-2 Protein Expression in Malignant Melanoma Cell Line

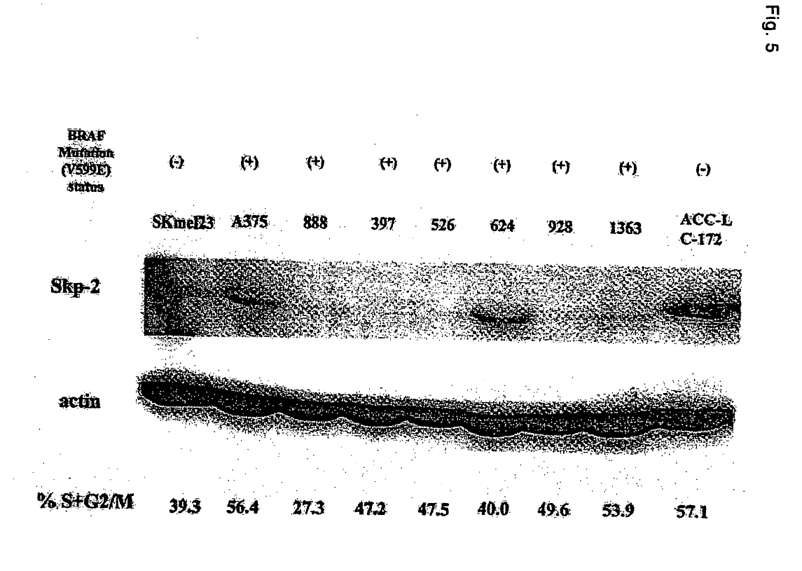

Proteins were extracted from 8 kinds of malignant melanoma cell lines (Skmel23, A375, 888, 397, 526, 624, 928, 1363) and ACC-LC-172 cells as a control, and the expression of Skp-2 protein was analyzed by Western blot method. The high expression of Skp-2 was observed in 3 kinds of malignant melanoma cell lines, Skmel23, A375mel and 624mel. ACC-LC-172, which is a small cell lung cancer cell line with high expression of Skp-2, was used as a positive control. Actin was blotted as a loading control for protein amount. The results are shown in FIG. 5, with the presence or absence of BRAF point mutation (V599E) and the cell cycle at the time of protein extraction (percentage (%) S+G2/M phase).

### EXAMPLE 15

### Cell Proliferation Suppression Effect of Simultaneous RNAi of BRAF and Skp-2 on Malignant Melanoma Cell Lines, and its Influence on p27^{Kip1} Protein

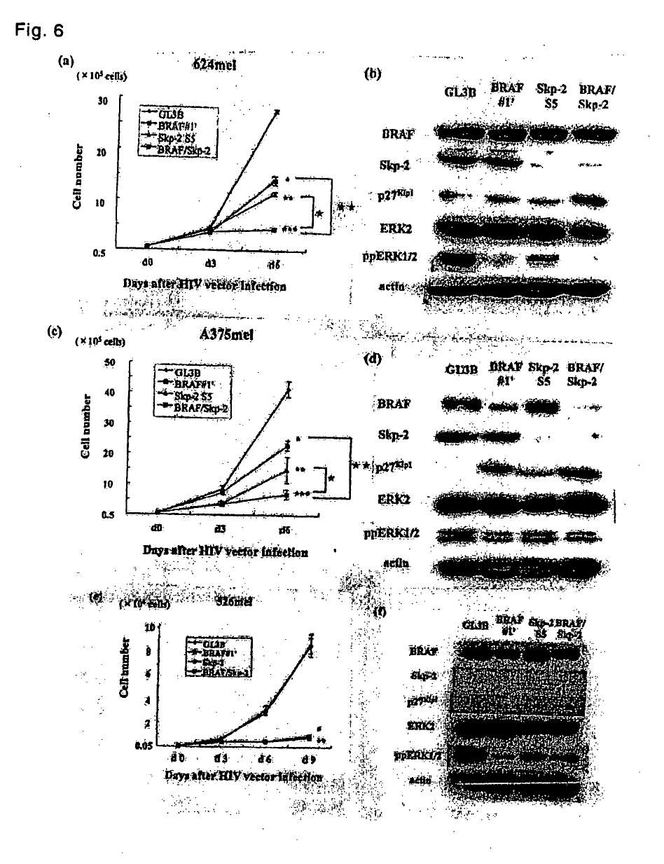

Though BRAF siRNA #1′ does not have the proliferation suppression effect on 293T cells which do not express the V599E-mutation-positive BRAF, and on Skmel23 with no V599E mutation, it has a prominent proliferation suppression effect on A375mel cell line, etc., having the mutation-positive BRAF. Therefore, it specifically suppresses the expression of the V599E-mutation-positive BRAF (see Japanese patent application No. 2004-124485). Two malignant melanoma cell lines (624mel, A375mel) that exhibited the high expression of Skp-2 in Example 14 and one malignant melanoma cell line (526mel) that did not exhibit high expression of Skp-2 in Example 14, all of which have BRAF point mutation (V599E), were infected with 4 kinds of HIV lentiviral vectors, control GL3B, BRAF siRNA #1′, Skp-2 siRNA S5, or BRAF/Skp-2 siRNA, at 100 MOI, and the number of the cells was counted every 3 days by trypan blue dye exclusion method until day 6 or 9 (n=3). The results are shown in FIG. 6. It can be seen from FIG. 6 that in the 2 malignant melanoma cell lines (624mel, A375mel) having BRAF point mutation (V599E) and exhibiting high expression of Skp-2, cell proliferation and cell invasive ability are suppressed when suppressing BRAF (V599E) and Skp-2 expression by RNAi simultaneously.

FIGS. 6(b), (d), (f) show the results of Western blot analysis of proteins extracted at the time of final observation corresponding to FIGS. (a), (c) and (e), respectively. The suppression of phosphorylated ERK and of Skp-2, caused by BRAF #1′ and Skp-2 S5, respectively, were observed. With regard to 624mel and A375mel, in the BRAF/Skp-2 simultaneous suppression group, both of them were observed, and the increase in p27^{Kip1} expression was greater than that in each of the single suppression groups. It is shown that: though the p27^{Kip1} expression level is thought to be independently controlled by BRAF-MAPK pathway and Skp-2-ubiquitine-proteasome pathway, when different active sites are knocked down simultaneously, more powerful recovery of p27^{Kip1} expression than the case where single active site is knocked down is observed, and as a result, more powerful cell proliferation suppression effect is obtained. As both of BRAF RNAi and Skp-2 RNAi act selectively only on cancer cells having the abnormality thereof, they have a possibility to be more powerful therapy while maintaining their specificity.

### EXAMPLE 16

### Matrigen Invasion Assay in A375mel Cell Line

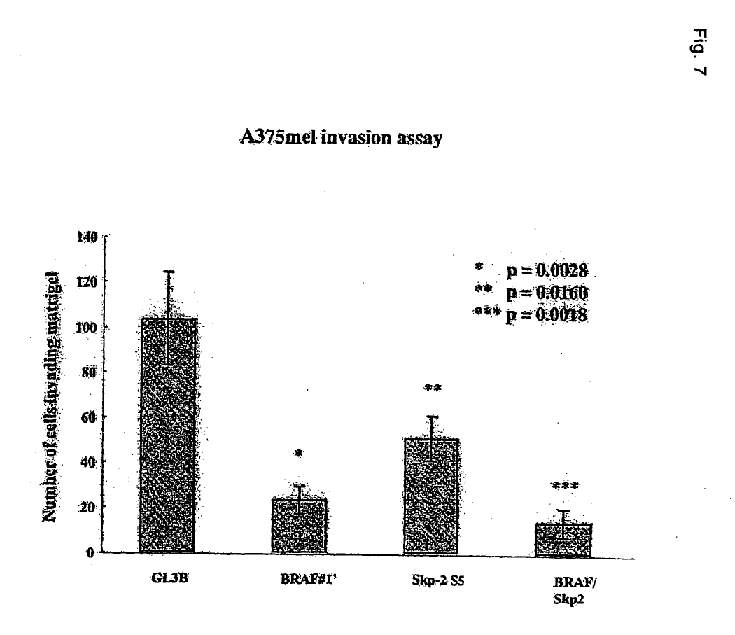

In a manner similar to that in Example 15, A375mel cells (2.5×10⁴ cells) infected with 4 kinds of HIV lentiviral vectors were seeded onto a matrigel invasion chamber (Bekton-Dickinson), and 22 hours later, the number of cells that moved to the back side of the chamber was counted. The results are shown in FIG. 7. From FIG. 7, it can be seen that the proliferation suppresion effect of 624mel, which was fairly limited in the case of BRAF RNAi alone, Skp-2 RNAi alone, was dramatically enhanced by using BRAF RNAi and Skp-2 RNAi together.

### EXAMPLE 17

### Discussion

When the knock down of Skp-2 in small cell lung cancer cell lines with the increased Skp-2 expression was attempted by virus-mediated RNAi, the sustained expression of Skp-2-specific siRNA suppressed the expression of Skp-2 almost completely, and induced the increase in p27^{Kip1} and the decrease in in vitro proliferation. Therefore, in this cell line, it is suggested that the increased Skp-2 expression could possibly be associated with the disorder of cell cycle regulation (overproliferation) caused by the hyperdegradation of p27^{Kip1}. From the observed facts mentioned above, it is suggested that Skp-2 could be a novel candidate target for gene therapy and/or molecular target therapy. Importantly, the proliferation of cells with low Skp-2 level such as 293T cells is hardly affected by Skp-2 RNAi, and the possibility that the inactivation of Skp-2 leads to a safe and selective therapy for cancers having the overexpression of Skp-2 is suggested.

The control of p21 level is mainly achieved by a transcriptional regulation mechanism. However, it is reported that the disruption of proteolysis that follows Skp-2-mediated ubiquitination is also involved in the control of p21 level (J. Biol. Chem, 278:25752-25757,2003). The present inventors have also found that p21 level increases after Skp-2 RNAi is conducted, and this is consistent with the above-mentioned report. Though the increase in p21 was not very prominent as the increase in p27^{Kip1} level, it is considered to be involved in the control of cell cycle whose regulation is disordered.

Further, by using intratumoral injection of Skp-2 siRNA adenoviral vector, an in vivo treatment animal model was constructed, and in vivo therapeutic effect was proved. Based on this, the possibility of gene therapy by siRNA vector targeting Skp-2 is suggested.

On the other hand, it has been already reported that the activation of MAPK pathway promotes the transfer of p27^{Kip1} from nucleus to cytoplasm and promotes the degradation of p27^{Kip1} by cytosol protease indirectly, and as a result, it leads to cell proliferation. The present inventors have reported the suppression effect of BRAF (V599E)-specific RNAi on the proliferation/invasive ability of cancer cell line (malignant melanoma cell line) with activated MAPK pathway associated with BRAF (V599E) mutation, and this method also acts on the recovery of p27^{Kip1} expression. Therefore, by constructing HIV vectors which express siRNAs for BRAF (V599E) and Skp-2 simultaneously in malignant melanoma cell lines having BRAF (V599E) mutation and exhibiting the overexpression of Skp-2 simultaneously, the influence of suppressing both expressions simultaneously was compared with that of single suppression groups, respectively. In 2 kinds of cell lines, cell proliferation and cell invasive ability were more significantly suppressed with reproducibility in the simultaneous suppression group than in each of single suppression groups (p27^{Kip1} is known to have a function to interfere RhoA signaling pathway to enhance cell motility). As the observed expression of p27^{Kip1} was stronger than that in each of single suppression groups, it is considered that the effect of improving malignant characters is observed via the recovery of stronger p27^{Kip1} expression. The BRAF mutation and the increased Skp-2 expression are gene abnormalities commonly observed in many cancers, therefore, the technology to suppress both of them simultaneously is expected to show stronger effect of cancer treatment on cancers having these mutations simultaneously.

### BRIEF DESCRIPTION OF DRAWINGS

[FIG. 1] This is a view showing that gene introduction of Skp-2 siRNA into small cell lung cancer cell line with the increased Skp-2 expression induces the inhibition of in vitro cell proliferation and the increase in p27^{Kip1} and p21 proteins. (a) In vitro cell proliferation assay. ACC-LC-172 cells (1×10⁵ cells) were infected with siRNA HIV vectors specific to control firefly luciferase (GL3B) or siRNA HIV vectors specific to Skp-2 mRNA (S2 and S5) at 100 MOI on day 0, then the number of living cells was counted by trypan blue dye exclusion method on day 3, 6 and 9. The vertical bars indicate the standard deviation of 3 experiments (*; P=0.0005, **; P<0.0001). This is one representative example of 3 experiments with similar results. (b) Western blot analysis. Proteins were extracted from the cells infected with siRNA HIV vectors 9 days after the infection. The GFP expression level at the time of protein extraction was comparable among 3 groups (98.7% to 99.9%). It was observed that the Skp-2 protein levels in S2- and S5-infected groups were significantly lowered in comparison to GL3B group. On the contrary, the p27^{Kip1} and p21 protein levels were increased in S2- and S5-infected groups. The p27^{Kip1} and p21 protein levels exhibited an inverse correlation with the Skp-2 protein level.

[FIG. 2] This is a view showing that the introduction of siRNA into 293T cells with no increased Skp-2 expression hardly affected on in vitro cell proliferation. (a) In vitro proliferation assay. 293T cells (3×10⁴ cells) were infected with control GLB3 or S5 vectors at 100 MOI on day 0, then the number of the cells was counted in the same manner as in FIG. 1(a). The vertical bars indicate the standard deviation of 3 experiments (*; P=0.1835). This is one representative example of 3 experiments with similar results. (b) Western blot analysis. Proteins were prepared in a same manner as in FIG. 1(b). The GFP expression level at the time of protein extraction was comparable between 2 groups (GL3B; 95.4%, S5; 100.0%). It was observed that the Skp-2 protein level in the S5-infected group was lowered in comparison to the GL3B group. On the other hand, the p27^{Kip1} protein level in the S5-infected group was increased in comparison to the GL3B group, however, it was weaker increase than the change in ACC-LC-172 cells. (c) Comparison of Skp-2 protein levels between 293T and ACC-LC-172. Skp-2 protein was significantly lower in 293T cells than in ACC-LC-172 cells. The percentages of S+G2/M in 293T cells and ACC-LC-172 cells at the time of protein extraction were 74.0% and 58.4%, respectively.

[FIG. 3] This is a view showing that the increase in Skp-2 protein did not affect on the enhancement of myc transcriptional activity of ACC-LC-172 cells. ACC-LC-172 cells stably expressing Skp-2-specific siRNA (siRNA S5) or BRAF-specific siRNA were transfected with 1 μg of pRL-SV40 (Renilla luciferase expression plasmid), and 1 μg of pGL3-hTERT, pGL3-Basic or pGL3-control (firefly luciferase expression plasmid guided by different promoters) by using Lipofectamine. 48 hours after the transfection, the cells were collected and both of the Renilla and firefly luciferase activities were measured to calculate each of firefly luciferase activities normalized by Renilla luciferase activity. It was observed that firefly luciferase activity in the cells transfected with pGL3-hTERT was increased by 1.6-fold of that of the cells transfected with pGL3-Basic, however, the suppression of the activity was not observed when Skp-2-specific siRNA was expressed. Each bar indicates mean value of 3 assays, and error bar indicates standard deviation. This is one representative example of 3 experiments with similar results.

[FIG. 4] This is a view showing the in vivo therapeutic effect of intratumoral injection of siRNA adenoviral vector specific to Skp-2. (a) Inhibitory effect of siRNA adenoviral vector specific to Skp-2 on Skp-2 protein. With the use of protein extracted from ACC-LC-172 cells infected with AdF35-Skp-2 siRNA S5 or AdF35-GL3B at 1 or 5 MOI, the Skp-2 protein level was quantitated. The Skp-2 level was significantly inhibited when the cells were infected with AdF35-Skp-2 siRNA S5 at 5 MOI. (b) In vivo tumor proliferation inhibitory effect of the introduction of Skp-2 siRNA via adenoviral vectors. Into ACC-LC-172 cells subcutaneously transplanted onto NOD/SCID mice, 1×10⁸ ifu of AdF35-Skp-2 siRNA S5 (n=5) or AdF35-GL3B (control) (n=4) was intratumorly injected 3 times every 2 days, and subsequently, the tumor volumes in 2 groups were compared in time course. *; p<0.05, and the vertical bar indicates standard deviation. [FIG. 5] This is a view showing the analysis results of Skp-2 protein expression in 8 kinds of malignant melanoma cell lines.

[FIG. 6] This is a view showing the proliferation suppression effect of simultaneous RNAi of BRAF and Skp-2 in malignant melanoma cell lines, and the influence on p27^{Kip1} protein. (a) In 624mel, *, **, and *** indicate p=0.0024, p=0.0005, and p=0.0002 (comparison to control GL3B), respectively, and [symbol] and [symbol] indicate p=0.0025, and p=0.0049, respectively. (c) In A375mel, *, **, and *** indicate p=0.0005, p=0.0008, and p<0.0001 (comparison to control GL3B), respectively, and [symbol] and [symbol] indicate p=0.0375, and p=0.0002, respectively. (e) In 526mel, *, and ** indicate p=0.0072, and p=0.0075 (all unpaired student t-test), respectively. No difference was observed in the introduction efficiency viewed as a ratio of GFP-positive at the time of final observation among each group. Each experiment was repeated 2 or 3 times to confirm reproducibility. The results of Western blot analysis of proteins extracted at the time of final observation corresponding to (a), (c) and (e) are shown in (b), (d) and (f), respectively.

[FIG. 7] This is a view showing the results of matrigel invasion assay in the A375mel cell line.

1. A double-stranded RNA capable of suppressing expression of Skp-2 gene, which consists of a sense strand RNA and an antisense strand RNA homologous to a specific sequence of Skp-2 mRNA, which is a target of the double-stranded RNA.

2. The double-stranded RNA according to claim 1, wherein the specific sequence of Skp-2 mRNA, which is a target of the double-stranded RNA consists of a RNA corresponding to the base sequence shown by SEQ ID NO: 2 of the sequence listing and a complementary sequence of the RNA.

3. The double-stranded RNA according to claim 1, wherein the specific sequence of Skp-2 mRNA which is a target of the double-stranded RNA consists of a RNA corresponding to the base sequence shown by SEQ ID NO: 3 of the sequence listing and a complementary sequence of the RNA.

4. The double-stranded RNA according to any one of claims 1 to 3, wherein the specific sequence of Skp-2 mRNA which is a target of the double-stranded RNA is a base sequence of 19 to 24 bp.

5. A double-stranded RNA expression cassette capable of expressing the double-stranded RNA according to any one of claims 1 to 4, which consists of a sense strand DNA—a linker—an antisense strand DNA of a specific sequence of Skp-2 gene.

6. The double-stranded RNA expression cassette according to claim 5, which consists of the base sequence shown by SEQ ID NO: 4 of the sequence listing.

7. The double-stranded RNA expression cassette according to claim 5, which consists of the base sequence shown by SEQ ID NO: 5 of the sequence listing.

8. A double-stranded RNA expression vector wherein the double-stranded RNA expression cassette according to any one of claims 5 to 7 is connected to downstream of a promoter.

9. The double-stranded RNA expression vector according to claim 8, which is an HIV lentiviral vector or an adenoviral vector.

10. A suppressor of Skp-2 gene expression comprising the double-stranded RNA according to any one of claims 1 to 4, the double-stranded RNA expression cassette according to any one of claims 5 to 7, or the double-stranded RNA expression vector according to claim 8 or 9, as an active component.

11. A suppressor of Skp-2 gene and mutated BRAF gene expression comprising the following (1) and (2) as active components:
(1) the double-stranded RNA according to any one of claims 1 to 4, the double-stranded RNA expression cassette according to any one of claims 5 to 7, or the double-stranded RNA expression vector according to claim 8 or 9;
(2) a double-stranded RNA capable of suppressing expression of mutated BRAF gene, which consists of a sense strand RNA and an antisense strand RNA homologous to a specific sequence of BRAF mRNA which is a target of the double-stranded RNA, a double-stranded RNA expression cassette capable of expressing the double-stranded RNA, which consists of a sense strand DNA—a linker—an antisense strand DNA of a specific sequence of BRAF gene, or a double-stranded RNA expression vector wherein the double-stranded RNA expression cassette is connected to downstream of a promoter.

12. A drug for preventing and/or treating a cancer which comprises the double-stranded RNA according to any one of claims 1 to 4, the double-stranded RNA expression cassette according to any one of claims 5 to 7, or the double-stranded RNA expression vector according to claim 8 or 9, as an active component.

13. The drug for preventing and/or treating a cancer according to claim 12, wherein the cancer results from overexpression of Skp-2 gene or is accompanied by overexpression of Skp-2 gene.

14. The drug for preventing and/or treating a cancer according to claim 12, wherein the cancer is a small cell lung cancer (SCLC).

15. A drug for preventing and/or treating a cancer which comprises the following (1) and (2) as active components:
(1) the double-stranded RNA according to any one of claims 1 to 4, the double-stranded RNA expression cassette according to any one of claims 5 to 7, or the double-stranded RNA expression vector according to claim 8 or 9;
(2) a double-stranded RNA capable of suppressing expression of mutated BRAF gene, which consists of a sense strand RNA and an antisense strand RNA homologous to a specific sequence of BRAF mRNA which is a target of the double-stranded RNA, a double-stranded RNA expression cassette capable of expressing the double-stranded RNA, which consists of a sense strand DNA—a linker—an antisense strand DNA of a specific sequence of BRAF gene, or a double-stranded RNA expression vector wherein the double-stranded RNA expression cassette is connected to downstream of a promoter.

16. A method for suppressing Skp-2 gene expression wherein the double-stranded RNA according to any one of claims 1 to 4, the double-stranded RNA expression cassette according to any one of claims 5 to 7, or the double-stranded RNA expression vector according to claim 8 or 9 is introduced into a living body, a tissue or a cell.

17. A method for suppressing Skp-2 gene and mutated BRAF gene expression wherein the following (1) and (2) are introduced into a living body, a tissue or a cell:
(1) the double-stranded RNA according to any one of claims 1 to 4, the double-stranded RNA expression cassette according to any one of claims 5 to 7, or the double-stranded RNA expression vector according to claim 8 or 9;
(2) a double-stranded RNA capable of suppressing expression of mutated BRAF gene, which consists of a sense strand RNA and an antisense strand RNA homologous to a specific sequence of BRAF mRNA which is a target of the double-stranded RNA, a double-stranded RNA expression cassette capable of expressing the double-stranded RNA, which consists of a sense strand DNA—a linker—an antisense strand DNA of a specific sequence of BRAF gene, or a double-stranded RNA expression vector wherein the double-stranded RNA expression cassette is connected to downstream of a promoter.

18. A method for preventing and/or treating a cancer wherein the double-stranded RNA according to any one of claims 1 to 4, the double-stranded RNA expression cassette according to any one of claims 5 to 7, or the double-stranded RNA expression vector according to claim 8 or 9 is introduced into a living body, a tissue or a cell.

19. The method for preventing and/or treating a cancer according to claim 18, wherein the cancer results from mutation or overexpression of Skp-2 gene or is accompanied by overexpression of Skp-2 gene.

20. The method for preventing and/or treating a cancer according to claim 18, wherein the cancer is a small cell lung cancer (SCLC).

21. A method for preventing and/or treating a cancer wherein the following (1) and (2) are introduced into a living body, a tissue or a cell:
(1) the double-stranded RNA according to any one of claims 1 to 4, the double-stranded RNA expression cassette according to any one of claims 5 to 7, or the double-stranded RNA expression vector according to claim 8 or 9;
(2) a double-stranded RNA capable of suppressing the expression of mutated BRAF gene, which consists of a sense strand RNA and an antisense strand RNA homologous to a specific sequence of BRAF mRNA, which is a target of the double-stranded RNA, a double-stranded RNA expression cassette capable of expressing the double-stranded RNA, which consists of a sense strand DNA—a linker—an antisense strand DNA of a specific sequence of BRAF gene, or a double-stranded RNA expression vector wherein the double-stranded RNA expression cassette is connected to downstream of a promoter.


SEQUENCE LISTING (16 sequences)

SEQ ID NO: 1 - DNA, 1357 bp, Homo sapiens
cagctctgcaagtttaatgcacgtattttaaactcccgggcctgcggacgctatgcacaggaagcacctccaggagattccagacctgagtagcaacgttgccaccagcttcacgtggggatgggattccagcaagacttctgaactgctgtcaggcatgggggtctccgccctggagaaagaggagcccgacagtgagaacatcccccaggaactgctctcaaacctgggccacccggagagccccccacggaaacggctgaagagcaaagggagtgacaaagactttgtgattgtccgcaggcctaagctaaatcgagagaactttccaggtgtttcatgggactcccttccggatgagctgctcttgggaatcttttcctgtctgtgcctccctgagctgctaaaggtctctggtgtttgtaagaggtggtatcgcctagcgtctgatgagtctctatggcagaccttagacctcacaggtaaaaatctgcacccggatgtgactggtcggttgctgtctcaaggggtgattgccttccgctgcccacgatcatttatggaccaaccattggctgaacatttcagcccttttcgtgtacagcacatggacctatcgaactcagttatagaagtgtccaccctccacggcatactgtctcagtgttccaagttgcagaatctaagcctggaaggcctgcggctttcggatcccattgtcaatactctcgcaaaaaactcaaatttagtgcgacttaacctttctgggtgttctggattctctgaatttgccctgcagactttgctaagcagctgttccagactggatgagctgaacctctcctggtgttttgatttcactgaaaagcatgtacaggtggctgttgcgcatgtgtcagagaccatcacccagctgaatcttagcggctacagaaagaatctccagaaatcagatctctctactttagttagaagatgccccaatcttgtccatctagacttaagtgatagtgtcatgctaaagaatgactgctttcaggaatttttccagctcaactacctccaacacctatcactcagtcggtgctatgatataatacctgaaactttacttgaacttggagaaattcccacactaaaaacactacaagtttttggaatcgtgccagatggtacccttcaactgttaaaggaagcccttcctcatctacagattaattgctcccatttcaccaccattgccaggccaactattggcaacaaaaagaaccaggagatatggggcatcaaatgccgactgacactgcaaaagcccagttgtctatgaagtatttattgcaggatggtgtctcttctt

SEQ ID NO: 2 - DNA, 18 bp, Homo sapiens
atcagatctctctactta

SEQ ID NO: 3 - DNA, 18 bp, Homo sapiens
aggtctctggtgttgtaa

SEQ ID NO: 4 - DNA, 49 bp, Homo sapiens
atcagatctctctacttattcaagagataagtagagagatctgattttt

SEQ ID NO: 5 - DNA, 49 bp, Homo sapiens
aggtctctggtgttgtaattcaagagattacaacaccagagaccttttt

SEQ ID NO: 6 - DNA, 19 bp, Artificial Sequence
  GL3BsiRNA HIV vector
gtgvgvtgvtggtgccaac

SEQ ID NO: 7 - DNA, 20 bp, Artificial Sequence
  BRAFsiRNA HIV vector
gctacagagaatctcvatgg

SEQ ID NO: 8 - DNA, 25 bp, Artificial Sequence
  forward primer
cgctggggccctcgctggcgtccct

SEQ ID NO: 9 - DNA, 25 bp, Artificial Sequence
  reverse primer
cagcggcagcacctcgcggtagtgg

SEQ ID NO: 10 - DNA, 2510 bp, Homo sapiens
cgcctcccggccccctccccgcccgacagcggccgctcgggccccggctctcggttataagatggcggcgctgagcggtggcggtggtggcggcgcggagccgggccaggctctgttcaacggggacatggagcccgaggccggcgccggccggcccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagagaaatctcgatggagtgggtcccatcagtttgaacagttgtctggatccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtggaaaaaaaaaaaaaaaaaaaaaaaaaaaaaaaaaaaaaaaccc

SEQ ID NO: 11 - DNA, 19 bp, Artificial Sequence
  siRNA#1′
gctacagagaaatctcgat

SEQ ID NO: 12 - DNA, 20 bp, Artificial Sequence
  siRNA#7′
gccacaactggctattgtta

SEQ ID NO: 13 - DNA, 21 bp, Artificial Sequence
  siDNA#10
gtatcaccatctccatatcat

SEQ ID NO: 14 - DNA, 52 bp, Artificial Sequence
  1′SENSE-LIKE-ANTISENSE-TTTTT
gctacagagaaatctcgatttcaagagaatcgagatttctctgtagcttttt

SEQ ID NO: 15 - DNA, 54 bp, Artificial Sequence
  7′SENSE-LINKER-ANTISENSE-TTTTT
gccacaactggctattgttattcaagagataacaatagccagttgtggcttttt

SEQ ID NO: 16 - DNA, 56 bp, Artificial Sequence
  10SENSE-LINKER-ANTISENSE-TTTTT
gtatcaccatctccatatcatttcaagagaatgatatggagatggtgatacttttt